(19) United States
(12) Patent Application Publication
(10) Pub. No.: US 2003/0003512 A1
(43) Pub. Date: Jan. 2, 2003

(54) Directed enzymatic modification of analytes for affinity capture and analysis

(75) Inventor: Howard Schulman, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SURROMED, INC, Mountain View, CA

(21) Appl. No.: 10/185,424

(22) Filed: Jun. 27, 2002

Related U.S. Application Data
(60) Provisional application No. 60/301,683, filed on Jun. 27, 2001.

(51) Int. Cl. G01N 33/53; G01N 33/542

(52) U.S. Cl. 435/7.1; 435/7.9

(57) ABSTRACT

Proteins are selectively extracted from complex biological mixtures for subsequent analysis by methods such as mass spectrometry. Protein-binding (e.g., PDZ or SH2) or cellular compartmentalizing (e.g., membrane-targeting) domains are provided to target specific proteins, and targeted proteins are enzymatically modified by attachment of modification molecules. For example, targeted proteins can be biotinylated by the enzyme biotin protein ligase. The protein-targeting domain and protein-modifying enzyme can be immobilized on a solid surface (macroscopic planar surface or particle surface), provided as a fusion protein, or chemically coupled. The modified proteins are then exposed to complementary molecules (e.g., avidin or streptavidin) that bind strongly to the modification molecule, resulting in affinity capture of the selected proteins. Because the protein-targeting domain brings the protein in proximity to the active site of the enzyme, the method effectively broadens the substrate specificity of the enzyme, allowing affinity capture of any desired protein subset.

### CROSS-REFERENCE TO RELATED APPLICATIONS

This application claims the benefit of U.S. Provisional Application No. 60/301,683, “Targeted Biotinylation for Affinity Capture in Multiplexed and Quantitative Mass Spectrometry of Analytes,” filed Jun. 27, 2001, incorporated herein by reference.

### FIELD OF THE INVENTION

The present invention relates generally to qualitative and quantitative analysis of components of biological mixtures. More particularly, it relates to a method for selectively enzymatically modifying subsets of proteins in a mixture to enable their subsequent affinity capture and analysis by methods such as mass spectrometry.

### BACKGROUND OF THE INVENTION

Recent developments in electrospray ionization (ESI) mass spectrometry (MS) and matrix-assisted laser desorption ionization (MALDI) MS enable the quantification and structural elucidation of novel proteins in complex biological samples. Although MS can be used for multiplexed quantitative assays over a broad range in molecular weight, interpreting mass spectra becomes difficult when the samples contain a large number of components of similar mass. Furthermore, the wide concentration range of proteins in biological samples makes it difficult to detect low-abundance proteins, because high-abundance proteins overwhelm or saturate separation systems. These problems can be addressed by separating the sample in a manner that reduces the complexity of the proteome and simplifies the acquired mass spectra. One useful sample separation method is affinity capture, in which proteins are selectively extracted from solution onto a surface containing molecules with which the proteins interact. The surface can be a single surface (e.g., planar) to which the sample is applied or the surfaces of nano- or microparticles dispersed in the sample and recovered after capture. The captured proteins subsequently can be eluted from the surface (if necessary) for analysis by MS.

One common method for biochemical analyte capture involves coupling analytes to the vitamin biotin in a process termed biotinylation. Biotin binds strongly to the proteins avidin and streptavidin, and biotionylated molecules are easily captured by a surface having immobilized avidin or streptavidin molecules. The biotin-avidin and biotin-streptavidin complexes have extremely large association constants (Ka−10¹⁵ M⁻¹ for avidin and 10¹³ M⁻¹ for streptavidin), energetically equivalent to covalent bonds, and are stable over a wide range of temperature and pH. For these reasons, biotin is commonly employed for analyte capture.

Biotinylation is typically accomplished using a chemically active form of biotin to label exposed lysine residues on target proteins. Lysine is one of the most frequently occurring amino acids, and chemical biotinylation can, therefore, be used to biotinylate and capture essentially all proteins. Enzymatic biotinylation, in contrast, is a highly specific process mediated by the enzyme biotin protein ligase (BPL). BPL covalently attaches biotin to endogenous proteins including carboxylases, decarboxylases, and transcarboxylases and has only between one and five substrates in a given organism. As a result, enzymatic biotinylation is too specific a process to be used for selective protein capture. It would be beneficial to have a biotinylation method with a specificity between that of chemical and enzymatic biotinylation.

Classes of proteins can instead be captured by agents that bind to specific protein domains, eliminating the need for biotinylation. In general, these interactions are of much lower affinity than biotin-avidin or biotin-streptavidin. After the protein is captured, it is necessary either to wash the surface bearing the capture agent to remove unbound material or to sort and recover particles containing immobilized capture agent. While high-affinity interactions can withstand these processes, low- or medium-affinity interactions may not. Interactions with Ka=10⁶ M⁻¹ have a dissociation rate of less than one second for a single bound protein. This can be addressed to some degree by increasing the number of capture agents, thereby enabling recapture of released proteins, but the effect is insufficient to enable analysis. The dependence of protein retention on handling time also introduces an undesirable source of variability into the measurement. As a result, many existing protein-binding domains cannot be exploited for capture and analysis of protein classes.

There is still a need, therefore, for a method for selectively targeting and capturing proteins from a complex biological sample for qualitative and quantitative analysis.

### SUMMARY OF THE INVENTION

The present invention addresses this need by providing methods and reagents for selectively modifying proteins for capture and analysis. Any of a large number of protein classes can be targeted for extraction.

The invention provides a method for analyzing a biological sample containing proteins. The sample is contacted with a protein-modifying enzyme and a protein-targeting domain, which targets a subset of proteins in the sample. The protein-targeting domain can be a protein-binding domain, which binds the protein subset, or a protein-compartmentalizing domain, which targets cellular compartments such as membranes or nuclei. The protein-modifying enzyme catalyzes attachment of a modification molecule to the targeted protein to produce a modified protein, which is contacted with a set of surface-bound complementary molecules. The modification molecules associate with the complementary molecules, causing the modified proteins to bind to the surface. They can then be analyzed by, for example, mass spectrometry. The modification method can be performed in solution, on a solid surface, or inside a cell. The protein-modifying enzyme and protein-targeting domain can be presented in a number of different ways. For example, they can be immobilized to one or more solid surfaces, provided as a fusion protein, or chemically coupled.

Preferably, the affinity between the captured protein and the protein-targeting domain is less than the affinity between the modification and complementary molecules, so that the method effectively converts a low- or medium-affinity interaction into a high-affinity interaction. Protein-binding domains can be proteins, carbohydrates, lipids, or synthetic chemical compounds having affinity for one or more proteins.

In an alternative embodiment, different modification molecules having known mass differences are used, enabling relative quantification of proteins in different samples by mass spectrometry. In this case, the modification molecule has a mass selected in dependence upon the subsequent analysis step.

Preferably, the protein-modifying enzyme is biotin protein ligase (BPL), and more preferably recombinant Escherichia coli (E. coli) biotin protein ligase (BirA), which attaches biotin to the targeted protein. The complementary molecule to biotin is either avidin or streptavidin. Additionally, BPL can be genetically modified to have a broader substrate specificity than that of naturally-occurring BPL. For example, the naturally-occurring DNA-binding domain can be replaced by the protein-targeting domain, and a linker of predetermined length can be inserted between the substrate binding domain and protein-targeting domain to facilitate protein modification. Alternatively, the protein-modifying enzyme can be a glutaminase, the modification molecule a glutathione derivative, and the complementary molecule glutathione S-transferase. The modification and complementary molecules can also be complementary nucleic acid sequences. Suitable protein-binding domains include SH2 or phosphotyrosine, PDZ or C-terminal peptides that bind PDZ, and hormones or hormone receptors.

The present invention also provides a fusion protein containing a proteinmodifying enzyme and a protein-targeting domain. Preferably, the protein-modifying enzyme is BPL, and more preferably recombinant E. coli BPL. Alternatively, the protein-modifying enzyme is a glutaminase. The protein-targeting domain can be a cellular compartmentalizing domain, such as a membrane-targeting domain, or a protein-binding domain such as SH2 or phosphotyrosine, PDZ or a C-terminal peptide that binds PDZ, or a hormone or hormone receptor. The fusion protein can also contain a linker of predetermined length separating the protein-modifying enzyme and protein-targeting domain.

Also provided by the present invention is a modified biotin protein ligase enzyme containing a protein-targeting domain. Preferably, the enzyme is modified recombinant E. coli BPL. The protein-targeting domain can replace the naturally-occurring DNA-binding domain of BPL. The modified enzyme can also contain a linker of predetermined length separating the protein-modifying enzyme and protein-targeting domain. The protein-targeting domain can be a cellular compartmentalizing domain, such as a membrane-targeting domain, or a protein-binding domain such as SH2 or phosphotyrosine, PDZ or a C-terminal peptide that binds PDZ, or a hormone or hormone receptor.

### BRIEF DESCRIPTION OF THE FIGURES

FIGS. 1A-1D schematically illustrate a method of the invention for selectively modifying and capturing proteins from a complex biological sample, using a protein-modifying enzyme and protein-binding domain immobilized to adjacent regions of a solid surface.

FIG. 2 illustrates a fusion protein of the present invention containing a protein-modifying enzyme and a protein-targeting domain.

FIG. 3 illustrates a chemically coupled protein-modifying enzyme and protein-targeting domain, according to the present invention.

FIG. 4 illustrates a genetically modified biotin protein ligase, in which the DNA-binding domain has been replaced by a protein-targeting domain.

FIG. 5 illustrates a genetically modified biotin protein ligase, in which a linker has been inserted between the active site and protein-targeting domain.

### DETAILED DESCRIPTION OF THE INVENTION

The present invention provides a method for selectively isolating proteins from a biological sample containing proteins and other molecules. Isolated proteins can then be subjected to any qualitative or quantitative biological assay or analysis method. In the method, proteins of interest are captured after being modified by enzymatic attachment of a modification molecule that binds to a surface-bound capture agent. Virtually any desired protein can be modified and captured, and protein-targeting domains are employed to target proteins of interest for modification. In this way, the method can convert a relatively low- or medium-affinity interaction between the protein-targeting domain and protein to a high-affinity interaction between the modification molecule and capture agent. The method uses enzymes that are, in some cases, modified to alter or broaden their substrate specificity.

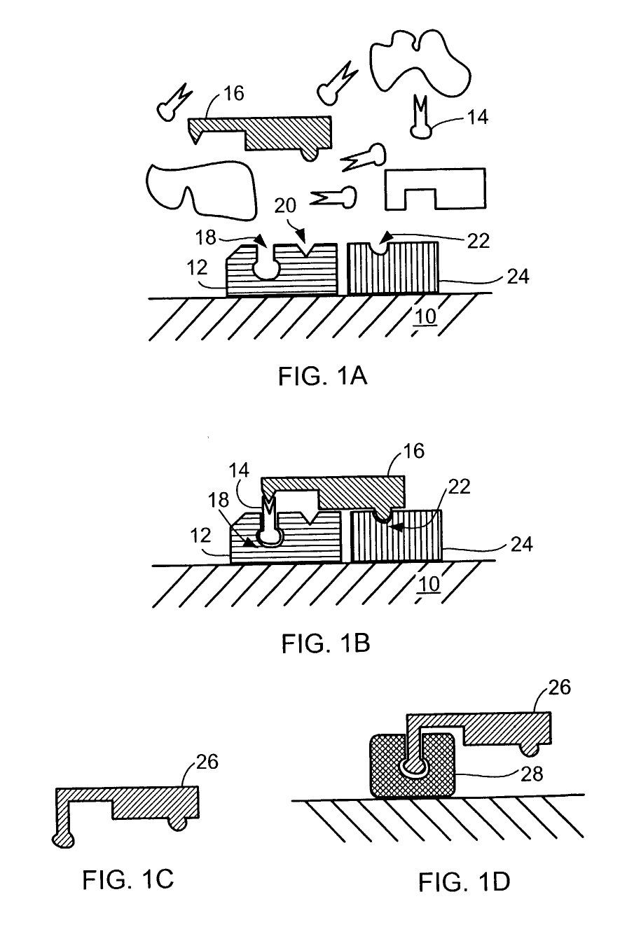

FIGS. 1A-1D schematically illustrate a general method of the invention for enzymatically modifying a subset of proteins. The figures are intended to show the concepts behind the invention, and the molecular shapes shown do not in any way limit the structures of the molecules involved. In FIG. 1A, a solid-phase surface 10 is provided having an immobilized protein-modifying enzyme 12 and an immobilized protein-targeting domain, in this case a protein-binding domain 22. The surface 10, which may be a planar surface, a well of a multi-well plate, the surface of a bead or nanoparticle, or other surface, is exposed to a sample containing proteins, a subset of which contains a protein 16. If the surface 10 is a single macroscopic surface, the sample is applied to the surface. If the surface 10 is one of a plurality of particle surfaces, the particles are dispersed within the sample and then subsequently recovered by suitable methods such as magnetic, density-based (e.g., centrifugation), or size-based (e.g., filtration) methods.

The protein-modifying enzyme 12 catalyzes the attachment, preferably covalent, of a modification molecule 14 to a protein, typically to a particular amino acid residue. The modification molecule 14 is either contained in the biological sample or added to the sample before or during exposure of the surface 10 to the sample. The protein-modifying enzyme 12 has at least one active site, a substrate-binding site 18. In FIG. 1A, the protein-modifying enzyme 12 has an additional active site 20 that does not participate in the attachment of the modification molecule 14 to a protein.

The protein-binding domain 22 is capable of binding one or more proteins, including, in this case, the protein 16. The protein-binding domain 22 may have a range of specificities, from highly specific to one protein, to specific to a particular amino acid, amino acid sequence, or structural feature occurring in a large number of proteins. Typically, the protein-binding domain 22 is part of a molecule 24, which is referred to as a targeting molecule 24. The sample can contain a large number of different proteins, and the protein-binding domain 22 is capable of binding a subset of the proteins.

As shown in FIG. 1B, the protein 16 is targeted by the targeting molecule 24 and binds to the protein-binding domain 22. As a result, an appropriate region of the protein 16 is in the vicinity of the active site 18 of the protein-modifying enzyme 12, and the modification molecule 14 becomes attached to the protein 16. Note that the protein 16 is not necessarily the intrinsic substrate of the protein-modifying enzyme 12. That is, when the protein-binding domain 22 is not employed to hold the protein 16 in the vicinity of the active site 18, the enzyme 12 may not catalyze modification of the protein 16. For this reason, the protein-binding domain 22, as employed by the present invention, is said to broaden the substrate specificity of the protein-modifying enzyme 12. However, the enzyme 12 may not be able to catalyze modification of all possible proteins, because it may be necessary for a particular amino acid residue to be in the vicinity of the active site 18. The result of the process depicted in FIG. 1B is that the modification molecule 14 is attached to the protein 16, resulting in a modified protein 26, shown in FIG. 1C. Of course, the method is typically performed with multiple enzymes 12, binding domains 22, and proteins 16, resulting in a set of modified proteins.

The sample is then introduced to a solid surface bearing a complementary molecule 28, preferably covalently linked to the surface, which can interact with the modified protein 26 via the modification molecule 14. This process is referred to as affinity capture. Any solid surface can be used, including a single planar surface, single non-planar surface, or multiple surfaces provided on particles distributed in solution or packed into a column. Such surfaces may be available commercially or prepared according to conventional methods. By virtue of the interaction between the modification molecule 14 and complementary molecule 28, the modified proteins can be isolated from the remainder of the sample. Only the proteins that were modified, i.e., those that bind to the protein-binding domain 22, can be captured using the complementary molecule 28. Typically, the affinity between the modification molecule 14 and complementary molecule 28 is much higher than that between the protein 16 and protein-binding domain 22, so that the method effectively converts a low-affinity interaction to a high-affinity interaction. Preferably, the modification molecule 14, protein 16, and complementary molecule 28 are selected so that the interaction between the modification molecule 14 and complementary molecule 28 is not significantly altered by the attachment of the modification molecule 14 to the protein 16.

In most cases, the modified protein 26 is separated from the enzyme 12 and binding domain 22 during affinity capture, simply as a result of the higher-affinity interaction between the modification molecule 14 and complementary molecule 28 as compared with that of the protein 16 and protein-binding domain 22. The targeting protein 24 can be removed by washing the surface bearing the complementary molecule 28 with a solution of suitable salt concentration. If necessary, conditions such as pH or temperature can be changed to facilitate de-binding.

After the desired proteins have been captured, as shown in FIG. 1D, they can be analyzed by any suitable method. The surface bearing the complementary molecule 28 is preferably washed and, for dispersed particles, recovered from solution before analysis. The binding of the modified protein 26 to the complementary molecule 28 is preferably maintained during wash and recovery steps. One example of analysis is protein identification or quantification by mass spectrometry (MS), including such methods as electrospray ionization (ESI), matrix-assisted laser desorption ionization (MALDI), or surface-enhanced laser desorption ionization (SELDI). The proteins 26 can be eluted from the complementary molecule 28 before analysis, or they can be presented to the analysis instrument while still bound to the complementary molecule 28. Any other desired analysis, such as assay of enzymatic activity, can be performed.

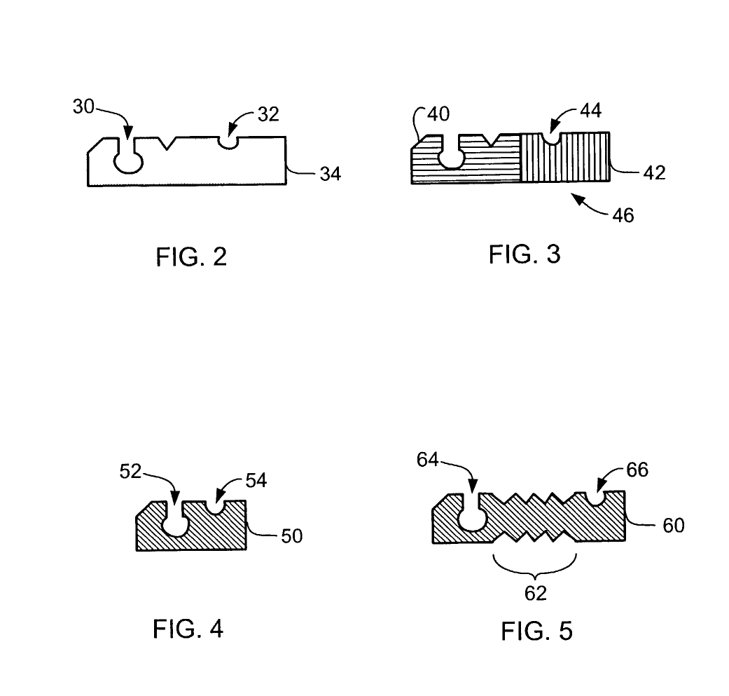

FIG. 2 illustrates an alternative embodiment of the invention, in which a protein-modifying enzyme 30 and a protein-targeting domain 32 are provided as a fusion protein 34. The fusion protein 34 can be prepared using conventional technology, i.e., by preparing a fusion construct containing nucleic acid sequences encoding the protein-modifying enzyme 30 and the protein-targeting domain 32 and introducing the fusion construct into a host cell that will express the protein. The fusion protein inherently provides the function of bringing the protein in proximity to the active site of the protein-modifying enzyme 30, and a solid phase surface is therefore unnecessary. A similar method to that illustrated in FIGS. 1A-1D can be used with the fusion protein 34 of FIG. 2. In this embodiment, however, the fusion protein 34 can be dispersed into the sample, where it targets the protein subset and catalyzes its modification. The solution is then exposed to one or more surfaces containing bound complementary molecules for affinity capture of the modified proteins.

The construct for the fusion protein can be introduced into a cell for in vivo enzymatic modification of a protein subset within the cell. The fusion protein is produced by the cell and catalyzes protein modification; the modified proteins are recovered after cell lysis. In this case, the modification molecule exists naturally in the cell. Although the proteins can instead be modified enzymatically after cell lysis, in vivo modification is preferred for some applications because the proteins are at higher concentration inside the cell than after lysis. The fusion protein can be introduced into cells by transfection of DNA or RNA by electroporation, by infection with a virus engineered to encode for the fusion protein, by microinjection of the catalytically active protein, or by using additional sequences that promote internalization. A number of sequences have been identified that penetrate cell membranes and internalize their associated cargo. For example, see M. Lidgren et al., “Cell-penetrating peptides,” Trends Pharmacol. Sci. 21:99-103 (2000), incorporated herein by reference.

The fusion protein 34 is not necessarily provided in solution. It may alternatively be immobilized on a solid surface. In this case, the enzymatic modification effected by the active site of the protein-modifying enzyme 30 can be of a protein bound to a protein-targeting domain of the same fusion protein or to a different (most likely adjacent) fusion protein. In general, having the fusion protein (or the distinct enzyme and targeting domain, as in the previous embodiment) immobilized on the solid surface is advantageous because it provides a high local concentration of both protein-binding domain and protein-modifying enzyme. Furthermore, there are many possible orientations of the two and thus a large possible number of modifiable proteins and manner in which a given protein can be modified.

Depending upon the particular protein-modifying enzyme 30, protein-targeting domain 32, and protein 16, it may be desirable to insert a linker molecule between the protein-modifying enzyme 30 and protein-targeting domain 32 to optimize catalytic activity of the protein-modifying enzyme 30 by optimizing access to the region of the protein 16 to be modified. The linker molecule has a length and structure selected so that the appropriate region of the protein is at an optimal distance and orientation to the catalytic site of the protein-modifying enzyme 30.

FIG. 3 illustrates a further alternative embodiment, in which a protein-binding enzyme 40 and a targeting molecule 42 with a protein-targeting domain 44 are chemically coupled to form a compound 46. Conventional methods can be used to couple the two chemically, depending upon their particular structure. As with the fusion protein 34 of FIG. 2, the chemically coupled compound 46 can be dispersed in the sample or immobilized on a solid surface to which the sample is added or which is distributed in the sample and then recovered.

When the protein-targeting domain is a protein-binding domain, any suitable protein-binding domain and targeting molecule can be employed. Protein-binding domains can be proteins, carbohydrates, lipids, or synthetic chemical compounds having affinity for one or more proteins. Ongoing research into protein-protein interactions continues to identify novel protein-binding domains, any of which can be employed in the present invention. When the protein-binding domains are proteins, they are typically independently folding modules of 35-150 amino acids that can be expressed in isolation from the host proteins while retaining their intrinsic ability to bind their partners. The domains can be organized into families related by sequence or by their ligand-binding properties. Additionally, the present invention can employ protein-binding domains that have been genetically engineered to have broader or narrower substrate specificity than the native proteins.

One suitable protein-binding domain family is an SH2 domain, which can be derived from a variety of proteins. SH2 domains interact with phosphotyrosines on distinct signaling molecules or classes of molecules. An SH2 domain can be used in the present invention to quantify tyrosine phosphorylation, an important post-translational modification, by capturing proteins containing phosphotyrosine residues. Different SH2 domains can be used to separate phosphotyrosine-containing proteins into different subclasses. Alternatively, the protein-binding domain can be a sequence containing tyrosine, which can be phosphorylated in vitro, to target specific subclasses of proteins containing SH2 domains.

As a second example, the protein-binding domain can be a PDZ domain, which binds to four or five C-terminal residues of particular proteins. Many protein scaffolds use enzymes or other proteins containing PDZ domains to bind proteins bearing the appropriate C-terminal sequence and thereby accelerate their assembly into complexes. As with SH2 domains, distinct PDZ domains enable fractionation into multiple protein subsets that interact with these domains. Alternatively, the proteinbinding domain can be a short peptide sequence with an appropriate C-terminal sequence that targets proteins bearing PDZ domains.

Other protein-binding families include, but are not limited to:

PTB domain, which binds phosphotyrosine

FHA and WW domains, which bind phosphoserine and phosphotyrosine

14-3-3 domain, which binds phosphoserine

bromodomain, which binds acetylated lysine

Additionally, the protein-binding domain can be a peptide hormone receptor or other receptor that binds peptides with high affinity. In this case, the receptor and a spacer or linker, if necessary, can be at the N- or C-terminal end of the catalytic domain. This embodiment is useful for capturing known receptors or receptors of orphan ligands (ligands of known biological activity whose receptors have not been identified).

The protein-binding domain can alternatively be a DNA-binding domain. For example, the native DNA-binding domain of BPL can be replaced with a different DNA-binding domain. This embodiment is useful for positioning the protein-modifying enzyme near transcriptional proteins or other DNA-binding proteins, which can then be modified.

In an alternative embodiment, the protein-targeting domain is a domain that is compartmentalized in a biological entity (e.g., cell) to facilitate enzymatic modification of the compartment. For example, the targeting domain targets membranes (isolated or of intact cells) via a lipid moiety or hydrophobic segment that inserts into a membrane. The lipid moiety can be chemically attached to the targeting molecule, or a site can be engineered that is normally modified by lipid, so that a lipid will become attached to the site. Alternatively, the protein-targeting domain is a nuclear localization signal that concentrates the modifying enzyme in a cell nucleus to selectively modify nuclear proteins. Other cellular compartments can be similarly targeted, as can the intracellular membrane surface by a fusion protein produced from DNA or RNA in cells. The compartmentalizing embodiments are preferably conducted using a fusion protein of protein-modifying enzyme and targeting domain or a chemically coupled enzyme and targeting domain.

In a preferred embodiment, the protein-modifying enzyme is biotin protein ligase (BPL), which catalyzes biotinylation of proteins, and more preferably recombinant Escherichia coli biotin protein ligase (BirA). However, other forms of BPL can be used. E. coli BPL and its amino acid sequence are described in A. Champman-Smith et al., “Molecular Biology of Biotin Attachment to Proteins,” J. Nutr. 129:477S-484S (1999), incorporated herein by reference, as are all of the references cited therein. The BirA protein is characterized by three active sites: a central catalytic domain for the protein to which biotin is bound, an N-terminal DNA-binding domain (the protein acts as the repressor of the biotin biosynthetic (bio) operon), and a C-terminal domain of unknown function.

When the protein-modifying enzyme is BPL, the modification molecule is biotin, and the complementary molecule is avidin or streptavidin. Solid phases (chips, microwell plates, or beads) bearing immobilized avidin or streptavidin are available commercially. BPL attaches biotin to lysine residues of specific proteins. In the present invention, the specificity of BPL is broadened so that biotin can be attached to lysine residues of proteins captured by the protein-binding domain.

In one embodiment, the specificity of BPL is broadened simply as a result of the protein-targeting domain's bringing the protein in close proximity to the active site of BPL, as shown in FIG. 1B. In this case, the protein is biotinylated at any accessible surface lysine residues.

In alternative embodiments of the invention, the specificity of BPL is broadened by genetically engineering the protein in a number of different ways. Although these embodiments will be discussed with reference to BPL, they can be performed with other protein-modifying enzymes. One genetic engineering embodiment, a genetically modified BPL protein 50, is illustrated in FIG. 4. The protein 50 contains the catalytic site 52 of the BirA protein. Additionally, it contains a protein-targeting domain 54 in place of the naturally-occurring DNA-binding domain of the BirA protein. This can be seen by comparing the protein 50 with the protein-modifying enzyme 12 of FIG. 1A and considering this enzyme 12 to be BirA. The protein 12 contains the catalytic site 18 and the DNA-binding site 20 (triangular). This DNA-binding site 20 is replaced by the protein-targeting domain 54 (semi-circular) in the protein 50. Note that in this embodiment, there is no need for an additional targeting protein 24 containing a protein-targeting domain 22. Rather, the protein 50 is analogous to the fusion protein 34 in that it contains both elements (protein-modifying enzyme and protein-targeting domain) necessary for the method. The protein 50 can be immobilized to a surface or dispersed in solution, or it can be introduced into a cell through transfection of a vector containing the appropriate DNA sequence. Conventional recombinant methods can be used to produce the protein 50 from a DNA construct encoding the protein 50.

FIG. 5 illustrates a related embodiment, a genetically engineered BPL protein 60 containing a linking sequence or linker 62 between the active site 64 and proteintargeting domain 66. The linking sequence varies in size and structure depending upon the protein-targeting domain 66 and the protein to be biotinylated. Conventional methods can be used to create a DNA construct encoding the protein with inserted linker 62 and modified targeting domain 66.

In an additional alternative embodiment of a genetically modified BPL, the intrinsic substrate specificity of the BirA or other native BPL protein is modified by modifying the catalytic site. In this embodiment, the genetically modified BPL serves as the protein-modifying enzyme only, and a distinct protein-targeting domain, as in FIGS. 1A-1D, 2, or 3, is preferably also provided.

The BirA protein biotinylates proteins by a two-stage reaction mechanism. In the first stage, the enzyme uses ATP and biotin to form the mixed anhydride biotinoyl-AMP, also called biotinoyl adenylate. Biotinoyl-AMP is sequestered in the enzyme's active site until the second stage of the reaction, attack of the anhydride by the amino group of a specific lysine residue of the acceptor protein to form an amide linkage.

In one type of modified substrate specificity, the active site residues involved in biotinoyl-AMP binding are modified so that the mutant active sites “leak” biotinoyl-AMP. These leaky mutants act as proximity-dependent biotinylation enzymes either by allowing non-cognate novel acceptor proteins to obtain access to the active site or by releasing biotinoyl-AMP to chemically biotinylate neighboring proteins. This can be performed by deleting amino acids within an N-terminal domain that acts as a lid on the active sites.

In general, site-directed mutagenesis, molecular evolution, or DNA shuffling techniques can be used to alter the binding site on BPL so that it can biotinylate a broad range of substrates. These techniques can also be used to alter the substrate specificity of any desired protein-modifying enzyme. As a result, selected non-physiological substrates can be modified based on the targeting molecules and protein-targeting domains used.

In one embodiment, it is possible to perform directed enzymatic modification of proteins without using the protein-targeting domain. The substrate specificity of the enzyme is altered in such a way as to permit modification of desired proteins only. By engineering the enzyme with many distinct substrate specificities, distinct subsets of substrates can be modified even without a protein-targeting domain.

In an additional embodiment, the protein-modifying enzyme is a transglutaminase, an enzyme that cross-links proteins, typically via glutamine and lysine residues. In the present invention, transglutaminases are used to attach modification molecules, in this case small molecules containing acceptor amines or primary alcohols, to glutamine residues of targeted proteins. The reaction involves a two-step acyl transfer reaction. First, the glutamine residue is activated by forming a thioester, resulting in release of ammonia from a γ-carboxamido intermediate. Next, the glutaminyl moiety is transferred to an acceptor amino group. Conventionally, this is the amino group of a protein-bound lysine, and a N-(γ-glutamyl)lysine isopeptide bond is produced. In the present invention, instead of the amino group of a protein, the glutaminyl moiety is transferred to an amino group of a small molecule. Transglutaminases have been shown to conjugate protein-bound glutamine with an acceptor amine to produce an isopeptide bond or with a primary alcohol to produce an ester, in Z. Nemes et al., “A novel function for transglutaminase 1: Attachment of long-chain ω-hydroxyceramides to involucrin by ester bond formation,” Proc. Natl. Acad. Sci. USA 96:8402-8407 (1999), incorporated herein by reference. Transglutaminases have also been shown to accept non-physiological primary amines such as poly(ethylene glycol) with a primary amine (II. Sato et al., “Transglutaminase-Mediated Dual and Site-Specific Incorporation of Poly(ethylene glycol) Derivatives into a Chimeric Interleukin-2,” Bioconjugate Chem. 11:502-509 (2000)) and 5-(biotinamido)pentylamine (T. F. Slaughter et al., “A microtiter plate transglutaminase assay utilizing 5-(biotinamido)pentylamine as substrate,” Anal. Biochem. 205:166-171 (1992)). Both of these references are incorporated herein by reference.

In embodiments employing a transglutaminase as the protein-modifying enzyme, a broader range of modification molecules can be used than in embodiments in which BPL is the protein-modifying enzyme. For example, transglutaminase can be used to biotinylate proteins at glutamine residues with modified forms of biotin. Any modification molecule having a primary amine or alcohol can be used, and it is preferable to select a molecule that has a high-affinity interaction with a complementary molecule. Suitable examples of modification and complementary molecule pairs include glutathione derivatives and glutathione-S-transferase or DNA sequences and complementary DNA sequences.

The present invention is not limited to the above-described protein modifications. For example, additional modifications include glycosylation, phosphorylation, prenylation, and ubiquitination (or ubiquitylation). Additionally, any suitable modification molecule and complementary molecule pair can be used. Preferable pairs have high binding affinity so that the molecules remain bound during washing and sorting steps.

The present invention can be used to modify and capture proteins in any desired biological sample. For example, typical samples include, but are not limited to, blood or other body fluids, intact cells (e.g., to target membrane-spanning proteins), distinct cellular compartments, whole extracts, or fractionated samples. In one implementation of the invention, the protein-binding domain targets membrane proteins, for example, via a lipid link or by inserting itself into the membrane. This is an effective way to selectively modify membrane proteins, because the local concentration of the protein-modifying enzyme is high.

In one embodiment of the invention, modification molecules with the same or similar structure but different masses are used to enable relative quantification of the same protein subset in different samples. This method is similar to mass tagging systems such as that disclosed in PCT Publication No. WO 02/42427, incorporated herein by reference, in which different samples are reacted with mass tags, structurally similar compounds that differ by a known mass amount. After reaction, the samples are combined and subjected to mass spectrometric analysis. Relative heights between spectral peaks differing by the known mass amount represent relative quantities of specific proteins in the samples. In the present invention, the modification molecules can have any variety of different masses, such as those resulting from different isotopes, different halides, or different homologous chemical substituents.

For example, biotin derivatives can be produced with deuterium replacing some of the stable hydrogens. Additionally, a number of different biotin derivatives have been produced, and there is considerable literature on synthesis and avidin binding of a variety of biotin derivatives. For example, see page 317 of M. Aslam and M. H. Dent, Eds., Bioconjugation: Protein Coupling Techniques for the Biomedical Sciences (1998), London: Macmillan References, Inc., incorporated herein by reference. Although the biotin derivatives can have any suitable modification, it is preferred that they retain high-affinity binding to avidin or streptavidin. High-affinity interaction involves almost the entire biotin molecule, particularly the imidazolidone ring, but a number of modifications, particularly in the valerate and thiophan moieties, retain high-affinity binding.

This embodiment can be extended to provide multiple modification molecules and multiple complementary molecules for use with the same sample, enabling distinct modification and capture of different analytes. For example, multiple cognate biotin/avidin pairs can be generated and used. Each variation of biotin is attached to a different protein subset for capture by a different avidin variant. The crystal structures of the biotin-avidin complex and the BPL-biotin complex are known and can be used to design appropriate biotin and avidin or streptavidin derivatives with sufficiently high binding affinities. Such an approach to designing complementary changes in ligands and enzymes has been demonstrated for kinases, G-proteins, and other proteins, as described in A. Bishop et at., “Unnatural ligands for engineered proteins: new tools for chemical genetics,” Annu. Rev. Biophys. Biomol. Struct. 29:577-606 (2000), incorporated herein by reference.

In another embodiment of the invention, the method is used to identify protein-protein interactions, e.g., for the purpose of understanding cellular metabolic pathways. In this embodiment, which can be performed in vitro or in intact cells such as mammalian cells, proteins interacting with the protein-targeting domain are selectively modified for capture. Subsequent analysis can determine the structure of the captured protein. This embodiment is particularly useful when conventional methods for identifying protein-protein interactions, such as yeast two-hybrid technology, are not optimal. For example, the present invention can be performed to target modified proteins that may not be properly modified and identified in a yeast cell.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for analyzing a sample containing proteins, comprising:
a) providing at least one protein-modifying enzyme and at least one protein-targeting domain;
b) contacting said protein-modifying enzyme and said protein-targeting domain with said sample, whereby a subset of said proteins interacts with said protein-targeting domain; and
c) contacting said protein-modifying enzyme and said protein-targeting domain with a modification molecule capable of associating with a complementary molecule, wherein said protein-modifying enzyme attaches said modification molecule to said subset of said proteins to form a set of modified proteins.

2. The method of claim 1, wherein said protein-modifying enzyme and said protein-targeting domain are immobilized to a solid surface.

3. The method of claim 1, further comprising contacting said modified proteins with at least one surface-bound complementary molecule, whereby said modification molecule of said modified proteins binds to said surface-bound complementary molecule.

4. The method of claim 1, further comprising analyzing said modified proteins.

5. The method of claim 4, wherein analyzing said modified proteins comprises performing mass spectrometry.

6. The method of claim 1, wherein a fusion protein comprises said protein-modifying enzyme and said protein-targeting domain.

7. The method of claim 1, wherein said protein-modifying enzyme and said protein-targeting domain are chemically coupled.

8. The method of claim 1, wherein said protein-modifying enzyme comprises biotin protein ligase and said modification molecule comprises biotin.

9. The method of claim 8, wherein said biotin-protein ligase is recombinant Escherichia coli biotin protein ligase.

10. The method of claim 8, wherein said complementary molecule is selected from the group consisting of avidin and streptavidin.

11. The method of claim 1, wherein said protein-modifying enzyme comprises glutaminase.

12. The method of claim 11, wherein said modification molecule comprises a glutathione derivative and said complementary molecule comprises glutathione S-transferase.

13. The method of claim 1, wherein said modification molecule comprises a nucleic acid sequence and said complementary molecule comprises a complementary nucleic acid sequence.

14. The method of claim 1, wherein said protein-targeting domain is a proteinbinding domain.

15. The method of claim 14, wherein an affinity between said subset of said proteins and said protein-binding domain is less than an affinity between said modification molecule and said complementary molecule.

16. The method of claim 14, wherein said protein-binding domain is selected from the group consisting of SH2 and phosphotyrosine.

17. The method of claim 14, wherein said protein-binding domain is selected from the group consisting of PDZ and a C-terminal peptide that binds PDZ.

18. The method of claim 14, wherein said protein-binding domain is selected from the group consisting of hormones and hormone receptors.

19. The method of claim 1, wherein said protein-targeting domain is a cellular compartmentalizing domain.

20. The method of claim 19, wherein said cellular compartmentalizing domain is a membrane-targeting domain.

21. The method of claim 1, wherein a genetically modified biotin protein ligase comprises at least one of said protein-modifying enzyme and said protein-targeting domain.

22. The method of claim 21, wherein said genetically modified biotin protein ligase has a broader substrate specificity than that of naturally-occurring biotin protein ligase.

23. The method of claim 21, wherein said genetically modified biotin protein ligase has a naturally-occurring DNA-binding domain replaced by said protein-targeting domain.

24. The method of claim 21, wherein said genetically modified biotin protein ligase comprises a linker of predetermined length separating said protein-targeting domain from a substrate-binding domain.

25. The method of claim 1, wherein said modification molecule has a mass selected in dependence upon a subsequent analysis step.

26. The method of claim 1, wherein steps (b) and (c) are performed within a cell.

27. A fusion protein comprising a protein-modifying enzyme and a protein-targeting domain.

28. The fusion protein of claim 27, wherein said protein-modifying enzyme comprises biotin protein ligase.

29. The fusion protein of claim 28, wherein said biotin-protein ligase is recombinant Escherichia coli biotin protein ligase.

30. The fusion protein of claim 27, wherein said protein-modifying enzyme comprises glutaminase.

31. The fusion protein of claim 27, wherein said protein-targeting domain is a cellular compartmentalizing domain.

32. The fusion protein of claim 27, wherein said protein-targeting domain is a protein-binding domain.

33. The fusion protein of claim 27, further comprising a linker of predetermined length separating said protein-modifying enzyme and said protein-targeting domain.

34. A modified biotin protein ligase enzyme comprising a protein-targeting domain.

35. The enzyme of claim 34, wherein said modified biotin protein ligase enzyme is modified recombinant Escherichia coli biotin protein ligase.

36. The enzyme of claim 34, wherein said protein-targeting domain is a cellular compartmentalizing domain.

37. The enzyme of claim 34, wherein said protein-targeting domain is a protein-binding domain.

38. The enzyme of claim 34, further comprising a linker of predetermined length separating said protein-targeting domain from a substrate-binding domain.
